(19) United States
(12) Patent Application Publication
(10) Pub. No.: US 2023/0212502 A1
(43) Pub. Date: Jul. 6, 2023

(54) NOVEL FERMENTATION SUBSTRATE FOR SOLID-STATE FERMENTATION

(71) Applicants: BAYER CROPSCIENCE BIOLOGICS GMBH, Wismar (DE); BAYER AKTIENGESELLSCHAET, Leverkusen (DE)

(72) Inventors: Bernhardt Michael STEINWENDER, Wismar (DE); Rakulan SIVANESAPILLAI, Wuppertal (DE); Ute EIBEN, Malchow/ Insel Poel (DE); Arite WOLF, Schwaan (DE); Ulrike HILSCHER, Wismar (DE); Peter LÜTH, Wismar (DE); Hans KORTE, Wismar (DE)

(73) Assignee: DANSTAR FERMENT AG, Zug (CH)

(21) Appl. No.: 18/000,113

(22) PCT Filed: May 26, 2021
(86) PCT No.: PCT/EP2021/063978

§ 371 Date: Nov. 28, 2022

(30) Foreign Application Priority Data
May 28, 2020 (EP) 20177000.5

(51) Int. Cl. C12N 1/14; C12N 1/20; C08L 3/02

(52) U.S. Cl. CPC C12N 1/14; C08L 3/02; C12N 1/20

(57) ABSTRACT

The present invention relates to a method for producing a composite fermentation substrate comprising a) Mixing of at least one thermoplastic with a starch containing, organic, granular or powdery, non-liquifiable growth medium; and b) Melt extruding the mixture obtained from a) into a desired shape. Furthermore, the invention relates to composite substrates produced according to the present method and a method for producing a microorganism, comprising (a) Providing a composite substrate according to the invention, (b) Inoculating the composite substrate with a microorganism to be cultivated; and (c) Incubating the composite substrate obtained from step (b) under controlled conditions.

Microorganisms have become a major source of substances which otherwise cannot at all be produced or only using complicated and costly chemical synthesis. Today, both microorganisms naturally occurring and producing a desired substance as well as genetically modified microorganisms are used. Most prominently, bacteria and fungi are used for such purposes.

Biological control agents based on microorganisms also become more and more important in the area of plant protection, be it for combatting various fungal or insect pests or for improving plant health. Although also viruses are available which can be used as biological control agents, mainly those based on bacteria and fungi are used in this area. The most prominent form of biological control agents based on fungi are the asexual spores called conidia as well as blastospores, but also other fungal propagules may be promising agents, such as (micro)sclerotia, ascospores, basidiospores, chlamydospores or hyphal fragments.

The production of fungi or fungal propagules has always been more difficult and time consuming than that of bacteria. Fungi require a more complex environment to grow efficiently. Solid-state fermentation of fungi, next to liquid fermentation, is the most promising method for the production of fungi. In recent times, promising progress in solid-state fermentation technology has been achieved; see e.g. WO2005/012478 or WO1999/057239. However, fungi are very demanding when it comes to a suitable substrate for cultivation. Accordingly, there is a need for substrates for solid-state fermentation which are easy to produce, cost effective and provide an increased yield of fungi or fungal propagules as compared to currently known fermentation substrates.

This technical problem has been solved as described in the following and according to the claims.

It is to be understood that preferred embodiments described under a specific aspect or embodiment of the present invention may equally be applied to different aspects or embodiments. For example, details relating to the method of producing a composite substrate may be applied to the composite substrates as well as for the uses disclosed herein. All embodiments concerning the characteristics of the present composite substrate as well as the method of producing such composite substrate are applicable to the respective other embodiment/aspect as well as to the uses or other methods disclosed herein.

Accordingly, the present invention relates to a method for producing a composite substrate comprising (a) Mixing of at least one thermoplastic with a starch comprising, organic, granular or powdery, non-liquifiable growth medium of plant origin; and (b) Melt extruding the mixture obtained from (a) into a desired shape.

A composite material is defined as a material made from two or more constituent materials with significantly different physical or chemical properties that, when combined, produce a material with characteristics different from the individual components. The individual components remain separate and distinct within the finished structure. Accordingly, a composite substrate denotes a composite material which is suitable for serving as a substrate for fermenting/producing microorganisms such as fungi.

Generally all thermoplastics may be used in the present invention, provided they can be processed, i.e. can be melt-extruded, in a temperature range of between about 60° C. and about 220° C. That means that for crystalline or partially crystalline thermoplastic polymers, the melting point (to be measured according to ISO 3146) should be significantly higher than the fermentation temperature of microorganism to be fermented using the resulting composite material (which, depending on the fungus, is usually between 10 and 40° C.) and not significantly lower than the temperature used for autoclaving the substrate prior to fermentation (which is usually between 100 and 135° C., preferably between 120° C. and 125° C., such as 121° C.), accordingly between about 100 and about 220° C., preferably, between about 120° C. and about 200° C., more preferably between about 122° C. and 200° C., such as between about 120° C. and about 180° C. or between 122° C. and about 180° C. For growth media that are not autoclaved, this requirement is omitted. Similarly, for amorphous thermoplastic polymers, the reference temperature is the glass transition temperature Tg, preferably the heat deflection temperature (HDT) instead of the melting temperature, with ranges as indicated above for the melting temperature of crystalline or partially crystalline thermoplastics. The heat deflection temperature is determined according to ISO 75-2 by applying 1.80 MPa, and the temperature is increased at 2° C./min. In the present invention, a thermoplastic is used for mechanical strength.

Some thermoplastics are more or less hydrolyzation stable based on ambient conditions, in particular the moisture content of the environment. Accordingly, hydrolyzation stable polymers are preferred in the present invention, thus, thermoplastics with low hydrolyzation stability are less suitable. However, said thermoplastics sensitive to hydrolyzation may be chosen under conditions where hydrolyzation stability is sufficient by adjusting e.g. the pH, extrusion temperature, surface area and/or possibly also potential residues of catalysts and/or monomers in the thermoplastic polymer. Finally, the glass transition temperature Tg of the thermoplastic should be below the melt-extrusion temperature.

The thermoplastic for example includes a polymer or copolymer of at least one ethylenically unsaturated monomer, the polymer or copolymer having repeating units provided with at least a polar group such as a hydroxy, alkoxy, carboxy, carboxyalkyl, alkyl carboxy, nitrile or acetal group. Preferred thermoplastics are composed of polyethylene, polyvinyl alcohol, polyacrylonitrile, ethylene-vinyl alcohol copolymer, ethylene-acrylic acid copolymer and other copolymers of an olefin selected from ethylene, propylene, isobutene and styrene with acrylic acid, vinyl alcohol, and/or vinyl acetate and mixtures thereof. The thermoplastic may also be a hydrophobic polymer, such as polyethylene, polypropylene and polystyrene.

Exemplary thermoplastics include olefins such as polyethylene (PE) or polypropylene (PP), polymethylmethacrylate (PMMA), acrylonitrile butadiene styrene (ABS), polyamides, polybenzimidazole, polycarbonates , polysulfone, polyoxymethylene (POM), polyether ether ketone (PEEK), polyetherimide, polyphenylene oxide, polyphenylene sulfide, thermoplastic polyesters such as polyethylene terephthalate (PET), polystyrene, polyurethane, polyvinyl chloride (PVC), polytetrafluoroethylene (PTFE) and a composition made from terephthalic acid dimethylester, 2,2,4,4-tetramethyl-1,3-cyclobutandiol and 1,4-cyclohexandimethanol (also commonly known as Tritan).

The polymers used may furthermore have a certain degree of crosslinking.

The organic growth medium of plant origin needs to comprise starch, e.g. in the form of native,pre-gelatinized or modified starch such as monostarch phosphate or hydroxypropylated starch. Besides its nutritional value, starch plays a critical role in that it decisively influences the morphology and mechanical properties of the extruded substrate. Specifically, adding heat, water and mechanical energy during extrusion leads to partial gelatinization of native starch granules. This process is generally referred to as cooking extrusion. When extruded under high die pressure gelatinized starch brings forth a porous structure of the composite substrate due to nucleation of steam bubbles with all advantages of the present invention as described. A porous structure may be realized as structure comprising open or closed pores or a mixture thereof, the term “porous” being defined as a solid structure comprising entities filled with gas. Herein water acts as both a plasticizer as well as blowing agent. Less volatile plasticizers with higher boiling points as compared to water such as glycerol, citric acid, fatty acids or polyols are preferred for cultivation periods of more than 30 days to decrease the degree of starch retrogradation and thereby adverse effects on the mechanical stability of the composite substrate.

The composite substrate according to the present invention is produced using melt extrusion after mixing thermoplastic and starch-comprising growth medium and optionally further components such as blowing and nucleating agents or plasticizers as described elsewhere as known in the art. It may be advantageous to premix two or more components prior to mixing all components, for example all components forming the growth medium. Preferably, mixing of thermoplastic and starch-comprising growth medium and optionally further components as described elsewhere takes place within the extrusion device using kneading elements for distributive mixing.

Commonly used extruders for extrusion of polymers include single screw and twin screw extruders. The latter are preferably used in the present invention in particular if the starch component is not pre-conditioned, that is present in its gelatinized form prior to extrusion. Twin screw extruders may also have co-rotating or counter-rotating screws. As extrusion should be effected under certain pressures to promote bubble nucleation and the pressure built up is usually higher in counter-rotating extruder screws, the latter are preferred. While the application of a single-screw is in principle possible it is unfavorable due to limited mixing efficiency and low pressure build-up. Suitable twin-screw extruders may involve co- or counter-rotating screws with intermeshing geometries as well as parallel or conical shapes. Counter-rotating twin-screw extruders generally lead to higher pressure build-up and are preferred for low-melt viscosity materials such as for low molecular weight thermoplastic binders or organic growth media with a high degree of gelatinization. Conical twin screw extruders are preferred for low bulk density powders so as to increase the feed volume in the feeding zone. For all other cases, co-rotating parallel twin-screw extruders with intermeshing screws equipped with (1) high intake screw elements in the feeding zone, (2) kneading elements close to a liquid injection port and (3) transport elements of decreasing pitch close to the die are preferred.

The preferred method of producing the compound substrate is extrusion using a twin-screw extruder. The extrusion process involves the following main steps: (1) Feeding granules of the thermoplastic binder, the starch containing organic growth medium and water into the extruder, (2) mixing the ingredients, (3) adding heat and mechanical power to melt the thermoplastic binder and induce gelatinization of native starch present in the organic growth medium, (4) pressure build-up towards the extruder die, (5) rapid expansion due to nucleation and growth of superheated steam bubbles after the material leaves the die and (6) cutting of the extrusion profile into granules of desired length. The production process is thus equivalent to the well-established food extrusion process.

The preferred method for feeding the granular of powder-based solid materials (thermoplastic and organic growth medium) into the extruder is by gravimetric screw feeding. If both materials exhibit comparable particle size distributions, they can be premixed prior to feeding from a single container as also indicated above. Otherwise, separate feeding is the preferred method.

Porous substrates with open pore-structure are preferred. Open pores facilitate rapid percolation of the substrate with germinable units of the microorganisms to be cultivated through capillary suction during inoculation of the fermenter. Porous substrates further exhibit a high specific surface area relative to bulk weight which facilitates access of microorganisms to the nutritional content of the substrate. In order to produce a porous substrate with open pore-structure, water is used as a blowing agent equivalent to the well-established food extrusion process. For ease of processing, either water is added through a liquid injection port close to the feed zone of the extruder at a water/solid mass flow ratio of up to 20 wt.-% or the residual moisture content of the starch comprising material of plant original is sufficiently high, such as at least 8 wt.-%, to provide for the amount of water necessary to serve as blowing agent for obtaining the desired, preferably porous, structure. As a further alternative besides the injection of water into the extruder barrel, preconditioning of the starch comprising material to a moisture content of at least 8 w% prior to extrusion is possible. Depending on the starch type and content of the organic growth medium, barrel temperature settings and specific power input, part of the excess water will be absorbed due to starch gelatinization. The remaining excess water remains unbound. Depending on temperature and pressure drop across the extruder die, the moisture content of the material will cause a rapid expansion as the material leaves the die due to nucleation and growth of superheated steam bubbles. In order to produce a porous substrate microstructure with high specific surface area, a head pressure close to the die of at least 2 MPa and preferably close to 65 MPa is required. For a given material composition, the head pressure is influenced by material throughput, screw speed, barrel temperature, die cross-sectional area and moisture content.

Accordingly, in a preferred embodiment, water is added as plasticizer and/or blowing agent in step a) of the present method. The amount of water added depends on the residual moisture content and the ratio of amylose and amylopectin in the starch and usually ranges between 8 and 30 wt.-% of the plant material, such as 10, 15, 20, 25 wt.-% or any value in between. Moisture content is determined using AACC method 44-15A (American Association of Cereal Chemists, 1983).

Suitable barrel temperatures in the melting zones of the extruder are generally limited by the melting or glass-transition temperature of the thermoplastic binder as lower limit and by the chemical degradation temperatures of the organic media (typically around 220-250° C.) as the upper limit, accordingly between 60° C. and about 220° C., preferably, between about 120° C. and about 220° C. depending on the amylose content in the starch. The barrel temperature close to the die should be higher than 100° C. so as to increase the superheated steam bubble growth. The barrel temperature in the liquid injection and mixing zones should be less than 100° C. to improve mixing and gelatinization processes.

Two ways of obtaining a composite fermentation substrate according to the invention are described in examples 1 and 2.

In the present invention, a thermoplastic is mixed with a growth medium of plant origin as described herein, and optionally further substances as described, and extruded. Plant material usually contains at least residual moisture which in the present invention serves as blowing agent to extrude the mixture into a preferably porous structure into a desired shape. Extrusion conditions vary with the thermoplastic and also with the plant material used. The present description and the examples provide ample guidance of how to select conditions in order to achieve the desired composite substrate by setting extrusion conditions.

Solid-state fermentation (SSF) is a method to grow predominantly filamentous fungi on a moist solid substrate. SSF can be carried out in two types of matrices, either in a natural substrate acting as solid substrate and a source of nutrients or a nutritionally inert support which must be impregnated with a liquid nutritive media. The most widely used substrates are of amilaceous or lignocellulosic origin. Substrates based on cereals are widely used in the fermentation of fungi used in agronomy.

In the course of the present invention, it was surprisingly found that an organic growth medium of plant origin and thermoplastic polymers may be combined to form a composite fermentation substrate for solid-state fermentation of fungal microorganisms. The present composite fermentation substrate shows very good mechanical stability which is necessary in order to withstand heat and moisture during the fermentation process which, depending on the fungal microorganism, may take several weeks. Such mechanical stability is also present if granular material such as cereal grains are chosen as growth medium. However, such granular growth media tend to agglomerate and tightly pack during fermentation which complicates efficient, continuous and equally distributed aeration of the fermentation chamber which has adverse effects on fungal growth and, consequently, on yield. On the other hand, materials based on isolated compounds of plant origin, such as starch or meal of different compositions, degrade rapidly and do not last sufficiently long to provide for a successful and completed fermentation run in many cases, thus such materials are not suitable to use as fermentations substrates.

Mechanical stability also after the fermentation is also an important feature as the fermented fungal microorganism needs to be harvested from the substrate. The fermentation substrate is required to remain mechanically stable also after fermentation, e.g. during harvesting process, which is accomplished with the present invention.

Finally, melt extrusion is an established form of processing which has become very cost-effective so that the present composite fermentation substrate can also be produced at low cost

The extrusion of step b) preferably takes place in a temperature range of between 120 and 220° C. The temperature depends on the thermoplastic used and its properties with regard to melting temperature (crystalline or partially crystalline polymers) or the glass transition temperature as well as the starch used and its moisture and amylose content. Accordingly, melt extrusion usually takes place at or around, preferably slightly above the melting temperature of a crystalline or partially crystalline thermoplastic polymer(s) or the HDT of an amorphous thermoplastic polymer(s) of choice all of which are known in the art.

The extrusion in step b) may take place with a Specific Mechanical Energy [SME] of between 50-300 Wh/kg.

The thermoplastic is preferably selected from the group consisting of polyolefins like linear or branched polypropylene or polyethylene (preferably of low, middle or high density structure like LLDPE, MDPE or HDPE), polyvinylchloride, polystyrene (such as high-impact polystyrene (HIPS)), polyurethane, polyacrylate, a derivative of any of the foregoing and copolymers of any of the foregoing.

More preferably, the thermoplastic is polypropylene.

The molecular weight of thermoplastic polymers suitable in the present invention, in particular of polypropylene, may vary depending on the polymer used and the above characteristic as concerning processability. The molecular weight (average molecular weight M_{w}, unless indicated otherwise), can be determined using gel permeation chromatography (GPC) having a polystyrol standard in DCM as solvent.

An indirect measure of molecular weight and also an appropriate characteristic of a thermoplastic to be used in the present invention is the Melt Flow Index (MFI) which is measured according to ISO 1133. For example for polypropylene, said MFI is measured at a temperature of 230° C. with an applied weight of 2.16 kg. An MFI of between 15 and 35 (g/10 min), preferably between 20 and 25, is regarded as appropriate for the thermoplastics to be used according to the present invention.

The shape of the composite substrate units resulting from the present method may be any shape producible using an extruder die. Preferably, the shape is selected from the group consisting of a polyhedron, a sphere or a part thereof, a donut, a cylinder, a cone, an ellipsoid, a paraboloid and a hyperboloid. Especially preferred are torus shapes (donut shapes).

In a preferred embodiment the shape in its longest dimension has a diameter of between 2 and 50 mm.

The ratio of thermoplastic:organic growth medium of step a) may range between 5:95 and 50:50. The individual ratio depends on the thermoplastic, the organic substrate and also on the fungus which is to grow on the resulting composite substrate. Accordingly, any ranges in between the above mentioned ones may be chosen. Exemplary ranges include (thermoplastic:organic growth medium) 10:90, 20:80, 30:70 and 40:60 and any range in between those ranges.

The organic growth medium to be used in the present method comprises starch because starch is a major nutrition source for most microorganisms such as fungi to be grown in the present method. Furthermore, upon extrusion, at least a part of the starch contained in the growth medium is converted from crystalline to amorphous starch. Without wishing to be bound by any scientific theory, Applicant hypothesizes that amorphous starch results in better bioavailability and digestibility of this carbon source for the fungus to be fermented. In addition, the present composite fermentation substrate makes said starch easily accessible to fungi by providing a porous, solid structure which can be colonized and consumed at the same time by the fungi during cultivation/fermentation. Organic growth medium comprising starch may preferably be or comprise materials of plant origin, such as plant fibers, e.g. originating from ground timber, cereals, parts of cereal grains, other plant parts and food waste both comprising polysaccharides; and mixtures of any of the foregoing.

Alternatively or in addition, said organic growth medium may comprise micro- and macronutrients or mixtures thereof, optionally in addition to the above-mentioned organic growth medium.

Preferably, the organic growth medium does not comprise isolated polysaccharide components but naturally occurring mixtures thereof with other components as present e.g. in cereals or plant fibers.

In one preferred embodiment, the growth media is a mixture of at least one component comprising starch and at least one more component which optionally comprises starch. In this embodiment, the starch comprising component may be isolated starch. Starch can be isolated from various plants, such as potatoes, rice, tapioca, maize, as well as cereals, such as rye, oats, wheat and the like. Maize starch is preferred. Preferably, the starch component has an amylopectin content of at least 65 % by weight, preferably at least 70% by weight. Chemically modified starches and starches of different genotypes can also be used. Still further, ethoxy derivatives of starch, hydrolyzed starch, starch acetates, cationic starches, cross-linked starches and the like may be used.

In a more preferred embodiment, the organic growth medium, in addition to isolated starch, comprises at least one component which comprises a further carbon source. Fungal microorganisms are able to utilize different carbon sources, depending on the fungal species. Besides species mainly feeding on starch, other species are able to digest cellulose or even lignin as carbon source. Accordingly, in some embodiments, the organic growth medium further comprises at least one component comprising cellulose and/or lignin.

Organic growth media to be used in the present method naturally have different moisture contents ranging from between 2 and 30%. Moisture in the form of water which is present as residual moisture in the medium itself or added is used as blowing agent or, in case of native starch, as a plasticizer in order to obtain a porous, foam-like structure. The moisture content has an influence on the pressure produced during extrusion and the resulting porosity and expansion ratio of the composite substrate.. Accordingly, the skilled person knows that pressure during extrusion need to be adapted to, inter alia, also the moisture content of the organic substrate, which may optionally be supplemented by additional water or further plasticizers as described herein.

It is preferred that at least one component of the organic growth medium is a cereal or based on a cereal. In this context, the term “based on” denotes that the organic growth medium originates from a cereal. For example, it may be an isolated compound or a mixture of compounds isolated from a cereal, such as cereal starch. Included within the term “based on” are also pieces of cereal grains (granulates) or cereal flower. Suitable cereals include wheat, rye, oat, rice, barley, maize, triticale, lentils, sorghum and soybean and mixtures thereof. Mixtures may e.g. comprise wheat and rye, wheat and maize, rye and maize, wheat and triticale, triticale and rye, triticale and maize, or even combinations of three or more of the foregoing.

The cereal may be used as grains or, preferably, coarsely ground or pulverized. For other plant parts comprising starch, such as plant parts also comprising plant fibers, it is preferred that they are coarsely ground or pulverized. Exemplary plant parts include corn cob grind, cereal brans including wheat and rye bran, legume fibers and wood fibers.

In another preferred embodiment, the organic growth medium comprises isolated starch and at least one further component based on cereal grains, including cereal grains, coarsely ground cereals, flower and malt.

The present composite fermentation substrate may furthermore comprise a further functional component, e.g. an alternative carbon source for fungal microorganisms, a nucleation agent, a blowing agent and/or a non-volatile plasticiser. For example, plant parts (further) comprising lignin and/or cellulose as described above may be used for fungal microogranisms which feed on such compounds.

Agents serving as nucleation agents may be e.g. mica, silicate, quartz, titanium dioxide, kaolin, amorphous silicic acids, magnesium carbonate, chalk, feldspar, barium sulfate, glass beads, ceramic beads, carbon fibers or glass fibers. In particular talc may serve as a nucleation agent in order to form water vapor and can accordingly be added to a maximum of about 2 wt.-%, preferably to about 1 wt.-%.

In a preferred embodiment, talc or a mineral filler based on talc is the sole reinforcing agent.

Suitable mineral fillers based on talc according to the invention are all particulate fillers which the person skilled in the art associates with talc. Similarly suitable are all particulate fillers which are commercially available and whose product descriptions contain the terms talc or talcum as characteristic features.

Preference is given to mineral fillers which have a content of talc according to DIN 55920 of greater than 50% by weight, preferably greater than 80% by weight, more preferably greater than 95% by weight and in particular greater than 98% by weight of the total mass of filler.

Talc is understood as meaning a naturally occurring or synthetically produced talc. Frequently used talc grades are characterized by a particularly high purity, characterized by an MgO content of from 28 to 35% by weight, preferably 30 to 33 wt.%, particularly preferably 30.5 to 32 wt.-% and an SiO2 content of 55 to 65 wt.%, preferably 58 to 64 wt.%, particularly preferably 60 to 62.5 wt .-%.

The preferred types of talc are further characterized by an A12 O3 content of less than 5 wt .-%, more preferably less than 1 wt. -%, in particular less than 0.7 wt .-% of the total mass. The use of the talc according to the invention in the form of finely ground types having an average particle size d50 of from. 0.1 to 5 µm, more preferably 0.7 to 2.5 µm, and particularly preferably 1.0 to 2.0 µm.

The talc-based mineral fillers to be used according to the invention preferably have an upper particle or particle size d95 of less than 10 µm, preferably less than 7 µm, more preferably less than 6 µm and particularly preferably less than 4.5 µm. The d95 and d50 values of the fillers are determined by sedimentation analysis with SEDIGRAPH D 5,000 according to ISO 13317-3. The talc-based mineral fillers may optionally be surface-treated in order to achieve a better coupling to achieve the polymer matrix. They can be equipped, for example, with a primer system based on functionalized silanes.

The composite substrate may further comprise a non-volatile plasticizer, e.g. to reduce the melt temperature of starch to prevent rapid re-crystallization of amorphous starch, i.e. retrogradation. Suitable plasticizers include glycerol and derivatives thereof such as glycerol triacetate, paraffin, sucrose acetate, xylitol, sorbitol, glycol, ethylene glycol and polypropylene adipate, citric acid, fatty acids, urea and formamide. Suitable amounts are up to 10 wt.-%, based on the starch content.

In a preferred embodiment, the thermoplastic in step a) is present as granulate or powder.

The invention also relates to a method for producing a microorganism comprising
(a) Providing a composite substrate which
   a. was produced by melt extruding a thermoplastic which is mixed with a starch containing organic, granular or powdery, non-liquefiable growth medium which (i) comprises plant fibers or (ii) is based on plants;
   b. comprises a melt extruded mixture of a thermoplastic and a starch containing organic, granular or powdery, non-liquefiable growth medium which (i) comprises plant fibers or (ii) is based on plants; or
   c. was obtained from a method comprising
      i. Mixing of at least one thermoplastic with a starch containing, organic, granular or powdery, non-liquifiable growth medium;
      ii. Melt extruding the mixture obtained from i) into a desired shape
(b) Inoculating the composite substrate with a microorganism to be cultivated; and
(c) Incubating the composite substrate obtained from step (b) under controlled conditions with all preferred embodiments as described herein.

The invention also relates to a composite substrate, produced by the methods described herein.

Furthermore, the present invention relates to a composite substrate, produced by extruding a thermoplastic which is mixed with a starch containing organic, granular or powdery, non-liquefiable growth medium which (i) comprises plant fibers or (ii) is based on plants. Preferred embodiments of this method are those to be found for the method of the present invention.

The invention also relates to a composite substrate comprising an extruded mixture of a thermoplastic and a starch containing organic, granular or powdery, non-liquefiable growth medium which (i) comprises plant fibers or (ii) is based on plants. Preferably, the present composite substrate is a solid-state fermentation substrate.

In any case, the present composite substrate is suitable for solid-state fermentation of microorganisms.

An exemplary embodiment of the composite substrate according to the present invention or produced by the method of the present invention, comprises
5-20 parts thermoplastic
30-95, preferably 50-90 parts of a starch containing organic, granular or powdery, non-liquefiable growth medium based on plants.

With the ingredients of the composite substrate of the invention being described in terms of parts, it is not necessary but preferred that it amounts to 100. The term “parts” relates to parts per weight.

As stated above, the present composite substrate may also comprise a mixture of different ingredients, such as more than one cereal or at least one cereal in more than one state (flower, granulate, part of the cereal (grain).

In particular embodiments of the present methods and composite substrates, the composite substrate comprises
5-20 parts thermoplastic
60-90 parts of a starch containing organic, granular or powdery, non-liquefiable growth medium which (i) comprises plant fibers or (ii) is based on plants. Preferably, the thermoplastic is polypropylene. The starch containing organic, granular or powdery, non-liquefiable substrate comprises preferably one more cereals, more preferably coarsely ground cereal. Even more preferably, at least one of the cereals is present in the form of malt. Especially preferred cereals in this embodiment are barley, rye and wheat. In one preferred embodiment, the cereal is barley. In another preferred embodiment, the cereal is rye. In yet another preferred embodiment, the cereal is wheat.

In a different embodiment, the present invention relates to e method for producing a microorganism, comprising
(a) Providing a composite substrate as disclosed herein or produced according to the method disclosed herein, inoculated with a microorganism to be cultivated;
(b) Incubating of the composite substrate obtained from step (a) under controlled conditions.

Method according to claim 18, wherein the microorganisms is a fungus, a yeast or a bacterium.

Fungi which may be used in the present method are those which are able to produce dormant fungal structures. Dormant fungal structures or organs in connection with the present invention include fungal spores such as conidia, ascospores, basidiospores, chlamydospores and blastospores as well as other dormant structures or organs such as sclerotia and microsclerotia in all stages of their development, i.e. during and after maturation. Preferably, the fungi produce exospores, more preferably conidia.

Fungi to be used in this method may be any fungus exerting a positive effect on plants such as a plant protective or plant growth promoting effect. Accordingly, said fungus may be an entomopathogenic fungus, a nematophagous fungus, a plant growth promoting fungus, a fungus active against plant pathogens such as bacteria or fungal plant pathogens, or a fungus with herbicidal action.

Exemplary species of plant growth/plant health supporting, promoting or stimulating fungi are E2.1 Talaromyces flavus, in particular strain V117b; E2.2 Trichoderma atroviride, in particular strain CNCM 1-1237 (e.g. Esquive® WP from Agrauxine, FR), strain SC1 described in International Application No. PCT/IT2008/000196), strain no. V08/002387, strain no. NMI No. V08/002388, strain no. NMI No. V08/002389, strain no. NMI No. V08/002390, strain LC52 (e.g. Sentinel from Agrimm Technologies Limited), strain kd (e.g. T-Gro from Andermatt Biocontrol), and/or strain LUI32 (e.g. Tenet from Agrimm Technologies Limited); E2.3 Trichoderma harzianum, in particular strain ITEM 908 or T-22 (e.g. Trianum-P from Koppert); E2.4 Myrothecium verrucaria, in particular strain AARC-0255 (e.g. DiTera™ from Valent Biosciences); E2.5 Penicillium bilaii, in particular strain ATCC 22348 (e.g. JumpStart® from Acceleron BioAg), and/or strain ATCC20851; E2.6 Pythium oligandrum, in particular strains DV74 or M1 (ATCC 38472; e.g. Polyversum from Bioprepraty, CZ); E2.7Rhizopogon amylopogon (e.g. comprised in Myco-Sol from Helena Chemical Company); E2.8 Rhizopogon fulvigleba (e.g. comprised in Myco-Sol from Helena Chemical Company); E2.9 Trichoderma harzianum, in particular strain TSTh20, strain KD, product Eco-T from Plant Health Products, ZA or strain 1295-22; E2.10 Trichoderma koningii; E2.11 Glomus aggregatum; E2.12 Glomus clarum; E2.13 Glomus deserticola; E2.14 Glomus etunicatum; E2.15 Glomus intraradices; E2.16 Glomus monosporum; E2.17 Glomus mosseae; E2.18 Laccaria bicolor; E2.19 Rhizopogon luteolus; E2.20 Rhizopogon tinctorus; E2.21 Rhizopogon villosulus; E2.22 Scleroderma cepa; E2.23 Suillus granulatus; E2.24 Suillus punctatapies; E2.25 Trichoderma virens, in particular strain GL-21; E2.26 Verticillium albo-atrum (formerly V. dahliae), in particular strain WCS850 (CBS 276.92; e.g. Dutch Trig from Tree Care Innovations); E2.27 Trichoderma asperellum, e.g. strain B35 (Pietr et al., 1993, Zesz. Nauk. A R w Szczecinie 161: 125-137) and E2.28 Purpureocillium lilacinum (previously known as Paecilomyces lilacinus) strain 251 (AGAL 89/030550; e.g. BioAct from Bayer CropScience Biologics GmbH).

In a more preferred embodiment, fungal strains having a beneficial effect on plant health and/or growth are selected from Talaromyces flavus, strain VII7b; Trichoderma harzianum strain KD or strain in product Eco-T from Plant Health Products, SZ; Myrothecium verrucaria strain AARC-0255; Penicillium bilaii strain ATCC 22348; Pythium oligandrum strain DV74 or M1 (ATCC 38472); Trichoderma asperellum strain B35; Trichoderma atroviride strain CNCM 1-1237 or strain SC1, and Purpureocillium lilacinum (previously known as Paecilomyces lilacinus) strain 251 (AGAL 89/030550).

In an even more preferred embodiment, fungal strains having a beneficial effect on plant health and/or growth are selected from Penicillium bilaii strain ATCC 22348, Trichoderma asperellum strain B35, Trichoderma atroviride strain CNCM 1-1237 or strain SC1 and Purpureocillium lilacinum (previously known as Paecilomyces lilacinus) strain 251 (AGAL 89/030550).

It is most preferred that the fungal strains having a beneficial effect on plant health and/or growth is Trichoderma asperellum strain B35, Trichoderma atroviride strain CNCM 1-1237 and/or Trichoderma atroviride strain SC1.

Bactericidally active fungi are e.g.: A2.2 Aureobasidium pullulans, in particular blastospores of strain DSM14940; A2.3 Aureobasidium pullulans, in particular blastospores of strain DSM 14941; A2.4 Aureobasidium pullulans, in particular mixtures of blastospores of strains DSM14940 and DSM14941; A2.9 Scleroderma citrinum.

Fungi active against fungal pathogens are e.g. B2.1 Coniothyrium minitans, in particular strain CON/M/91-8 (Accession No. DSM-9660; e.g. Contans® from Bayer CropScience Biologics GmbH); B2.2 Metschnikowia fructicola, in particular strain NRRL Y-30752; B2.3 Microsphaeropsis ochrace, in particular strain P130A (ATCC deposit 74412); B2.4Muscodor albus, in particular strain QST 20799 (Accession No. NRRL 30547); ; B2.5 Trichoderma harzianum rifai, in particular strain KRL-AG2 (also known as strain T-22, /ATCC 208479, e.g. PLANTSHIELD T-22G, Rootshield®, and TurfShield from BioWorks, US) and strain T39 (e.g. Trichodex® from Makhteshim, US); B2.6 Arthrobotrys dactyloides; B2.7 Arthrobotrys oligospora; B2.8 Arthrobotrys superba; B2.9 Aspergillus flavus, in particular strain NRRL 21882 (e.g. Afla-Guard® from Syngenta) or strain AF36 (e.g. AF36 from Arizona Cotton Research and Protection Council, US); B2.10 Gliocladium roseum (also known as Clonostachys rosea f. rosea), in particular strain 321U from Adjuvants Plus, strain ACM941 as disclosed in Xue (Efficacy of Clonostachys rosea strain ACM941 and fungicide seed treatments for controlling the root tot complex of field pea, Can Jour Plant Sci 83(3): 519-524), strain IK726 (Jensen DF, et al. Development of a biocontrol agent for plant disease control with special emphasis on the near commercial fungal antagonist Clonostachys rosea strain ‘IK726’; Australas Plant Pathol. 2007;36:95-101), strain 88-710 (WO2007/107000), strain CR7 (WO2015/035504)or strains CRrO, CRM and CRr2 disclosed in WO2017109802; B2.11 Phlebiopsis (or Phlebia or Peniophora) gigantea, in particular strain VRA 1835 (ATCC 90304), strain VRA 1984 (DSM16201), strain VRA 1985 (DSM16202), strain VRA 1986 (DSM16203), strain FOC PG B20/5 (IMI390096), strain FOC PG SP log6 (IMI390097), strain FOC PG SP log5 (IMI390098), strain FOC PG BU3 (IMI390099), strain FOC PG BU4 (IMI390100), strain FOC PG 410.3 (IMI390101), strain FOC PG 97/1062/116/1.1 (IMI390102), strain FOC PG B22/SP1287/3.1 (IMI390103), strain FOC PG SH1 (IMI390104) and/or strain FOC PGB22/SP1190/3.2 (IMI390105) (Phlebiopsis products are e.g. Rotstop® from Verdera and FIN, PG-Agromaster®, PG-Fungler®, PG-IBL®, PG-Poszwald® and Rotex® from e-nema, DE); B2.12 Pythium oligandrum, in particular strain DV74 or M1 (ATCC 38472; e.g. Polyversum from Bioprepraty, CZ); B2.13 Scleroderma citrinum; B2.14 Talaromyces flavus, in particular strain V117b; B2.15 Trichoderma asperellum, in particular strain ICC 012 from Isagro or strain SKT-1 (e.g. ECO-HOPE® from Kumiai Chemical Industry), strain T34 (e.g. ASPERELLO® from Biobest Group NV and T34 BIOCONTROL® by Biocontrol Technologies S.L., ES); B2.16 Trichoderma atroviride, in particular strain CNCM 1-1237 (e.g. Esquive® WP from Agrauxine, FR), strain SC1 described in International Application No. PCT/IT2008/000196), strain 77B (T77 from Andermatt Biocontrol), strain no. V08/002387, strain NMI no. V08/002388, strain NMI no. V08/002389, strain NMI no. V08/002390, strain LC52 (e.g. Sentinel from Agrimm Technologies Limited), strain LUI32 (e.g. Tenet by Agrimm Technologies Limited), strain ATCC 20476 (IMI 206040), strain T11 (IMI352941/ CECT20498), strain SKT-1 (FERM P-16510), strain SKT-2 (FERM P-16511), strain SKT-3 (FERM P-17021); B2.17 Trichoderma harmatum; ; B2.18 Trichoderma harzianum, in particular, strain KD, strain T-22 (e.g. Trianum-P from Koppert), strain TH35 (e.g. Root-Pro by Mycontrol), strain DB 103 (e.g. T-Gro 7456 by Dagutat Biolab); B2.19 Trichoderma virens (also known as Gliocladium virens), in particular strain GL-21 (e.g. SoilGard by Certis, US); B2.20 Trichoderma viride, in particular strain TV1(e.g. Trianum-P by Koppert), strain B35 (Pietr et al., 1993, Zesz. Nauk. A R w Szczecinie 161: 125-137); B2.21 Ampelomyces quisqualis, in particular strain AQ 10 (e.g. AQ 10® by CBC Europe, Italy); B2.22 Arkansas fungus 18, ARF; B2.23 Aureobasidium pullulans, in particular blastospores of strain DSM14940, blastospores of strain DSM 14941 or mixtures of blastospores of strains DSM14940 and DSM 14941 (e.g. Botector® by bio-ferm, CH); B2.24 Chaetomium cupreum (e.g. BIOKUPRUM TM by AgriLife); B2.25 Chaetomium globosum (e.g. Rivadiom by Rivale); B2.26 Cladosporium cladosporioides, in particular strain H39 (by Stichting Dienst Landbouwkundig Onderzoek); B2.27 Dactylaria candida; B2.28 Dilophosphora alopecuri (e.g. Twist Fungus); B2.29 Fusarium oxysporum, in particular strain Fo47 (e.g. Fusaclean by Natural Plant Protection); B2.30 Gliocladium catenulatum (Synonym: Clonostachys rosea f. catenulate), in particular strain J1446 (e.g. Prestop ® by Lallemand); B2.31 Lecanicillium lecanii (formerly known as Verticillium lecanii), in particular conidia of strain KV01 (e.g. Vertalec® by Koppert/Arysta); B2.32 Penicillium vermiculatum; ; B2.33 Trichoderma gamsii (formerly T. viride), in particular strain ICC080 (IMI CC 392151 CABI, e.g. BioDerma by AGROBIOSOL DE MEXICO, S.A. DE C.V.); B2.34 Trichoderma polysporum, in particular strain IMI 206039 (e.g. Binab TF WP by BINAB Bio-Innovation AB, Sweden); B2.35 Trichoderma stromaticum (e.g. Tricovab by Ceplac, Brazil); B2.36 Tsukamurella paurometabola, in particular strain C-924 (e.g. HeberNem®); B2.37 Ulocladium oudemansii, in particular strain HRU3 (e.g. Botry-Zen® by Botry-Zen Ltd, NZ); B2.38 Verticillium albo-atrum (formerly V. dahliae), in particular strain WCS850 (CBS 276.92; e.g. Dutch Trig by Tree Care Innovations); B2.39 Muscodor roseus, in particular strain A3-5 (Accession No. NRRL 30548); B2.40 Verticillium chlamydosporium; B2.41 mixtures of Trichoderma asperellum strain ICC 012 and Trichoderma gamsii strain ICC 080 (product known as e.g. BIO-TAM™ from Bayer CropScience LP, US), B2.42 Simplicillium lanosoniveum and B2.43 Trichoderma fertile (e.g. product TrichoPlus from BASF).

In a preferred embodiment, the biological control agent having fungicidal activity is selected from Coniothyrium minitans, in particular strain CON/M/91-8 (Accession No. DSM-9660)Aspergillus flavus, strain NRRL 21882 (available as Afla-Guard® from Syngenta) and strain AF36 (available as AF36 from Arizona Cotton Research and Protection Council, US); Gliocladium roseum strain 321U, strain ACM941, strain IK726strain 88-710 (WO2007/107000), strain CR7 (WO2015/035504); Gliocladium catenulatum strain J1446; Phlebiopsis (or Phlebia or Peniophora) gigantea, in particular the strains VRA 1835 (ATCC 90304), VRA 1984 (DSM16201), VRA 1985 (DSM16202), VRA 1986 (DSM16203), FOC PG B20/5 (IM1390096), FOC PG SP log6 (IMI390097), FOC PG SP log5 (IMI390098), FOC PG BU3 (IMI390099), FOC PG BU4 (IMI390100), FOC PG 410.3 (IMI390101), FOC PG 97/1062/116/1.1 (IMI390102), FOC PG B22/SP1287/3.1 (IMI390103), FOC PG SH1 (IM1390104), FOC PG B22/SP1190/3.2 (IMI390105) (available as Rotstop® from Verdera and FIN, PG-Agromaster®, PG-Fungler®, PG-IBL®, PG-Poszwald®, and Rotex® from e-nema, DE); Pythium oligandrum, strain DV74 or M1 (ATCC 38472) (available as Polyversum from Bioprepraty, CZ); Talaromyces flavus, strain VII7b; Ampelomyces quisqualis, in particular strain AQ 10 (available as AQ 10® by CBC Europe, Italy); Gliocladium catenulatum (Synonym: Clonostachys rosea f. catenulate) strain J1446, Cladosporium cladosporioides, e. g. strain H39 (by Stichting Dienst Landbouwkundig Onderzoek), Trichoderma virens (also known as Gliocladium virens), in particular strain GL-21 (e.g. SoilGard by Certis, US), Trichoderma atroviride strain CNCMI-1237, strain 77B, strain LU132 or strain SC1, having Accession No. CBS 122089, Trichoderma harzianum strain T-22 (e.g. Trianum-P from Andermatt Biocontrol or Koppert), Trichoderma asperellum strain SKT-1, having Accession No. FERM P-16510 or strain T34, Trichoderma viride strain B35 and Trichoderma asperelloides JM41R (Accession No. NRRL B-50759).

In a more preferred embodiment, the fungal species having fungicidal activity is selected from Coniothyrium minitans, in particular strain CON/M/91-8 (Accession No. DSM-9660) (available as Contans® from Prophyta, DE); Gliocladium roseum strain 321U, strain ACM941, strain IK726; Gliocladium catenulatum, in particular strain J1446; and Trichoderma virens (also known as Gliocladium virens), in particular strain GL-21. Said fungal species may also preferably be Coniothyrium minitans strain CON/M/91-8 (Accession No. DSM-9660) or Gliocladium catenulatum strain J1446, Trichoderma atroviride strain CNCM 1-1237, Trichoderma atroviride strain SC1 and Trichoderma viride strain B35.

Nematicidally active fungal species include D2.1Muscodor albus, in particular strain QST 20799 (Accession No. NRRL 30547); D2.2 Muscodor roseus, in particular strain A3-5 (Accession No. NRRL 30548); D2.3 Paecilomyces lilacinus (also known as Purpureocillium lilacinum), in particular P. lilacinus strain 251 (AGAL 89/030550; e.g. BioAct from Bayer CropScience Biologics GmbH); D2.4 Trichoderma koningii; D2.5 Harposporium anguillullae; D2.6 Hirsutella minnesotensis; D2.7 Monacrosporium cionopagum; D2.8 Monacrosporium psychrophilum; D2.9 Myrothecium verrucaria, in particular strain AARC-0255 (e.g. DiTeraTM by Valent Biosciences); D2.10 Paecilomyces variotii, strain Q-09 (e.g. Nemaquim® from Quimia, MX); D2.11 Stagonospora phaseoli (e.g. from Syngenta); D2.12 Trichoderma lignorum, in particular strain TL-0601 (e.g. Mycotric from Futureco Bioscience, ES); D2.13 Fusarium solani, strain Fs5; D2.14 Hirsutella rhossiliensis; D2.15 Monacrosporium drechsleri; D2.16 Monacrosporium gephyropagum; D2.17 Nematoctonus geogenius; D2.18 Nematoctonus leiosporus; D2.19 Neocosmospora vasinfecta; D2.20 Paraglomus sp, in particular Paraglomus brasilianum; D2.21 Pochonia chlamydosporia (also known as Vercillium chlamydosporium), in particular var. catenulata (IMI SD 187; e.g. KlamiC from The National Center of Animal and Plant Health (CENSA), CU); D2.22 Stagonospora heteroderae; D2.23 Meristacrum asterospermum, D2.24 Duddingtonia flagrans.

In a more preferred embodiment, fungal strains with nematicidal effect are selected from Paecilomyces lilacinus, in particular spores of P. lilacinus strain 251 (AGAL 89/030550) (available as BioAct from Bayer CropScience Biologics GmbH); Harposporium anguillullae; Hirsutella minnesotensis; Monacrosporium cionopagum; Monacrosporium psychrophilum; Myrothecium verrucaria, strain AARC-0255 (available as DiTeraTM by Valent Biosciences); Paecilomyces variotii; Stagonospora phaseoli (commercially available from Syngenta); and Duddingtonia flagrans.

In an even more preferred embodiment, fungal strains with nematicidal effect are selected from Paecilomyces lilacinus, in particular spores of P. lilacinus strain 251 (AGAL 89/030550) (available as BioAct from Bayer CropScience Biologics GmbH); and Duddingtonia flagrans.

Fungi active against insects (entomopathogenic fungi) include C2.1Muscodor albus, in particular strain QST 20799 (Accession No. NRRL 30547); C2.2 Muscodor roseus in particular strain A3-5 (Accession No. NRRL 30548); C2.3 Beauveria bassiana, in particular strain ATCC 74040 (e.g. Naturalis® from CBC Europe, Italy; Contego BB from Biological Solutions Ltd.; Racer from AgriLife); strain GHA (Accession No. ATCC74250; e.g. BotaniGuard Es and Mycontrol-O from Laverlam International Corporation); strain ATP02 (Accession No. DSM 24665); strain PPRI 5339 (e.g. BroadBand™ from BASF); strain PPRI 7315, strain R444 (e.g. Bb-Protec from Andermatt Biocontrol), strains IL197, IL12, IL236, IL10, IL131, IL116 (all referenced in Jaronski, 2007. Use of Entomopathogenic Fungi in Biological Pest Management, 2007: ISBN: 978-81-308-0192-6), strain Bv025 (see e.g. Garcia et al. 2006. Manejo Integrado de Plagas y Agroecologia (Costa Rica) No. 77); strain BaGPK; strain ICPE 279, strain CG 716 (e.g. BoveMax® from Novozymes); C2.4 Hirsutella citriformis; C2.5 Hirsutella thompsonii (e.g. Mycohit and ABTEC from Agro Bio-tech Research Centre, IN); C2.6 Lecanicillium lecanii (formerly known as Verticillium lecanii), in particular conidia of strain KV01 (e.g. Mycotal® and Vertalec® from Koppert/Arysta); C2.7 Lecanicillium lecanii (formerly known as Verticillium lecanii), in particular conidia of strain DAOM198499; C2.8 Lecanicillium lecanii (formerly known as Verticillium lecanii), in particular conidia of strain DAOM216596; C2.9 Lecanicillium muscarium (formerly Verticillium lecanii), in particular strain VE 6 / CABI(=IMI) 268317/ CBS 102071/ ARSEF5128 (e.g. Mycotal from Koppert); C2.10 Metarhizium acridum, e.g. ARSEF324 from GreenGuard by BASF or isolate IMI 330189 (ARSEF7486; e.g. Green Muscle by Biological Control Products); C2.11 Metarhizium anisopliae complex, e.g. strain Cb 15 (e.g. ATTRACAP® from BIOCARE); strain ESALQ 1037 (e.g. from Metarril® SP Organic), strain E-9 (e.g. from Metarril®SP Organic), strain M206077, strain C4-B (NRRL 30905), strain ESC1, strain 15013-1 (NRRL 67073), strain 3213-1 (NRRL 67074), strain C20091, strain C20092, strain F52 (DSM3884/ ATCC 90448; e.g. BIO 1020 by Bayer CropScience and also e.g. Met52 by Novozymes) or strain ICIPE 78; C2.15 Metarhizium robertsii 23013-3 (NRRL 67075); C2.13 Nomuraea rileyi; C2.14 Paecilomyces fumosoroseus (new: Isaria fumosorosea), in particular strains Apopka 97 (available as PreFeRal from Certis, USA), Fe9901 (available as NoFly from Natural industries, USA), ARSEF 3581, ARSEF 3302, ARSEF 2679 (ARS Collection of Entomopathogenic Fungal Cultures, Ithaca, USA), IfB01 (China Center for Type Culture Collection CCTCC M2012400), ESALQ1296, ESALQ1364, ESALQ1409 (ESALQ: University of São Paulo (Piracicaba, SP, Brazil)), CG1228 (EMBRAPA Genetic Resources and Biotechnology (Brasilia, DF, Brazil)), KCH J2 (Dymarska et al., 2017; PLoS one 12(10)): e0184885), HIB-19, HIB-23, HIB-29, HIB-30 (Gandarilla-Pacheco et al., 2018; Rev Argent Microbiol 50: 81-89), CHE-CNRCB 304, EH-511/3 (Flores-Villegas et al., 2016; Parasites & Vectors 2016 9:176 doi: 10.1186/s13071-016-1453-1), CHE-CNRCB 303, CHE-CNRCB 305, CHE-CNRCB 307 (Gallou et al., 2016; fungal biology 120 (2016) 414-423), EH-506/3, EH-503/3, EH-520/3, PFCAM, MBP, PSMB1 (National Center for Biololgical Control, Mexico; Castellanos-Moguel et al., 2013; Revista Mexicana De Micologia 38: 23-33, 2013), RCEF3304 (Meng et al., 2015; Genet Mol Biol. 2015 Jul-Sep; 38(3): 381-389), PF01-N10 (CCTCC No. M207088), CCM 8367 (Czech Collection of Microorganisms, Brno), SFP-198 (Kim et al., 2010; Wiley Online: DOI 10.1002/ps.2020), K3 (Yanagawa et al., 2015; J Chem Ecol. 2015; 41(12): 118-1126), CLO 55 (Ansari Ali et al., 2011; PLoS One. 2011; 6(1): e16108. DOI: 10.1371/journal.pone.0016108), IfTS01, IfTS02, IfTS07 (Dong et al. 2016 / PLoS ONE 11(5): e0156087. doi:10.1371/journal.pone.0156087), P1 (Sun Agro Biotech Research Centre, India), If-02, If-2.3, If-03 (Farooq and Freed, 2016; DOI: 10.1016/j.bjm.2016.06.002), Ifr AsC (Meyer et al., 2008; J. Invertebr. Pathol. 99:96-102. 10.1016/j.jip.2008.03.007), PC-013 (DSMZ 26931), P43A, PCC (Carrillo-Pérez et al., 2012; DOI 10.1007/s11274-012-1184-1), Pf04, Pf59, Pf109 (KimJun et al., 2013; Mycobiology 2013 Dec; 41(4): 221-224), FG340 (Han et al., 2014; DOI: 10.5941/MYCO.2014.42.4.385), Pfr1, Pfr8, Pfr9, Pfr10, Pfr11, Pfr12 (Angel-Sahagun et al., 2005; Journal of Insect Science), Ifr531 (Daniel and Wyss, 2009; DOI 10.1111/j.1439-0418.2009.01410.x), IF-1106 (Insect Ecology and Biocontrol Laboratory, Shanxi Agricultural University), I9602, 17284 (Hussain et al. 2016, DOI: 10.3390/ijms17091518), I03011 (Patent US 4618578), CNRCB1 (Centro Nacional de Referencia de Control Biologico (CNRCB), Colima, Mexico), SCAU-IFCF01 (Nian et al., 2015; DOI: 10.1002/ps.3977), PF01-N4 (Engineering Research Center of Biological Control, SCAU, Guangzhou, P. R. China) Pfr-612 (Institute of Biotechnology (IB-FCB-UANL), Mexico), Pf-Tim, Pf-Tiz, Pf-Hal, Pf-Tic (Chan-Cupul et al. 2013, DOI: 10.5897/AJMR12.493); C2.15 Aschersonia aleyrodis; C2.16 Beauveria brongniartii (e.g. Beaupro from Andermatt Biocontrol AG); C2.17 Conidiobolus obscurus; C2.18 Entomophthora virulenta (e.g. Vektor from Ecomic); C2.19 Lagenidium giganteum; C2.20 Metarhizium flavoviride; C2.21 Mucor haemelis (e.g. BioAvard from Indore Biotech Inputs & Research); C2.22 Pandora delphacis; C2.23 Sporothrix insectorum (e.g. Sporothrix Es from Biocerto, BR); C2.24 Zoophtora radicans.

In a preferred embodiment, fungal strains having an insecticidal effect may be selected from Beauveria bassiana, strain ATCC 74040 (available as Naturalis® from Intrachem Bio Italia), strain GHA (Accession No. ATCC74250) (available as BotaniGuard Es and Mycontrol-O from Laverlam International Corporation), strain ATP02 (Accession No. DSM 24665), strain CG 716 (available as BoveMax® from Novozymes), strains IL197, IL12, IL236, IL10, IL131, IL116 (all referenced in Jaronski, 2007. Use of Entomopathogenic Fungi in Biological Pest Management, 2007: ISBN: 978-81-308-0192-6), strain Bv025 (see e.g. Garcia et al. 2006. Manejo Integrado de Plagas y Agroecologia (Costa Rica) No. 77), and strain PPRI 5339 (e.g. BroadBand™ from BASF); Hirsutella citriformis; Hirsutella thompsonii (with some strains available as Mycohit and ABTEC from Agro Bio-tech Research Centre, IN); Lecanicillium lecanii (formerly known as Verticillium lecanii) conidia of strain KV01 (available as Mycotal® and Vertalec® from Koppert/Arysta); Lecanicillium lecanii (formerly known as Verticillium lecanii) conidia of strain strain DAOM198499; Lecanicillium lecanii (formerly known as Verticillium lecanii) conidia of strain DAOM216596; Lecanicillium muscarium (formerly Verticillium lecanii), strain VE 6 / CABI(=IMI) 268317/ CBS 102071/ ARSEF5128; Metarhizium brunneum, strain F52 (DSM3884/ ATCC 90448) (available as Met52 by Novozymes); M. acridum (ARSEF324 available as GreenGuard by BASF); M. acridum isolate IMI 330189 (ARSEF7486) (available as Green Muscle by Biological Control Products); Metarhizium brunneum strain Cb 15 (e.g. ATTRACAP® from BIOCARE); Nomuraea rileyi; Paecilomyces fumosoroseus (new: Isaria fumosorosea), strain apopka 97 or strain Fe9901; and Beauveria brongniartii (e.g. Beaupro from Andermatt Biocontrol AG).

In a more preferred embodiment, fungal strains having an insecticidal effect are selected from Beauveria bassiana, in particular strain ATCC 74040 (available as Naturalis® from Intrachem Bio Italia), strain GHA (Accession No. ATCC74250) (available as BotaniGuard Es and Mycontrol-O from Laverlam International Corporation), strain ATP02 (Accession No. DSM 24665), strain CG 716 (available as BoveMax® from Novozymes), strains IL197, IL12, IL236, IL10, IL131, IL116 (all referenced in Jaronski, 2007. Use of Entomopathogenic Fungi in Biological Pest Management, 2007: ISBN: 978-81-308-0192-6), strain Bv025 (see e.g. Garcia et al. 2006. Manejo Integrado de Plagas y Agroecologia (Costa Rica) No. 77); Paecilomyces fumosoroseus (new: Isaria fumosorosea), strain apopka 97 or strain Fe9901; Lecanicillium lecanii (formerly known as Verticillium lecanii), conidia of strain KV01 (available as Mycotal® and Vertalec® from Koppert/Arysta), conidia of strain strain DAOM198499 or conidia of strain DAOM216596; Metarhizium brunneum, strain F52 (DSM3884/ ATCC 90448) (available as Met52 by Novozymes); Metarhizium acridum, strain ARSEF324; Nomuraea rileyi; Lecanicillium muscarium (formerly Verticillium lecanii), strain VE 6 / CABI(=IMI) 268317/ CBS 102071/ ARSEF5128; and Beauveria brongniartii (e.g. Beaupro from Andermatt Biocontrol AG).

It is even more preferred that said fungus is a strain of the genus Metarhizium spp.. The genus Metahrizium comprises several species some of which have recently been re-classified (for an overview, see Bischoff et al., 2009; Mycologia 101 (4): 512-530). Members of the genus Metarhizium comprise M. pingshaense, M. anisopliae, M. robertsii, M. brunneum (these four are also referred to as Metarhizium anisopliae complex), M. acridum, M. majus, M. guizouense, M. lepidiotae and M.globosum. Of these, M. anisopliae, M. robertsii, M. brunneum and M. acridum are even more preferred, and those of M. brunneum and M. acridum are most preferred. Exemplary strains belonging to Metarhizium spp. which are also especially preferred are Metarhizium acridum ARSEF324 (product GreenGuard by BASF) or isolate IMI 330189 (ARSEF7486; e.g. Green Muscle by Biological Control Products); Metarhizium brunneum strain Cb 15 (e.g. ATTRACAP® from BIOCARE), or strain F52 (DSM3884/ ATCC 90448; e.g. BIO 1020 by Bayer CropScience and also e.g. Met52 by Novozymes); Metarhizium anisopliae complex strains ESALQ 1037 or strain ESALQ E-9 (both from Metarril® WP Organic), strain M206077, strain C4-B (NRRL 30905), strain ESC1, strain 15013-1 (NRRL 67073), strain 3213-1 (NRRL 67074), strain C20091, strain C20092, or strain ICIPE 78. Most preferred are isolate F52 (a.k.a. Met52) which primarily infects beetle larvae and which was originally developed for control of Otiorhynchus sulcatus. and ARSEF324 which is commercially used in locust control. Commercial products based on the F52 isolate are subcultures of the individual isolate F52 and are represented in several culture collections including: Julius Kühn-Institute for Biological Control (previously the BBA), Darmstadt, Germany: [as M.a. 43]; HRI, UK: [275-86 (acronyms V275 or KVL 275)]; KVL Denmark [KVL 99-112 (Ma 275 or V 275)]; Bayer, Germany [DSM 3884]; ATCC, USA [ATCC 90448]; USDA, Ithaca, USA [ARSEF 1095]. Granular and emulsifiable concentrate formulations based on this isolate have been developed by several companies and registered in the EU and North America (US and Canada) for use against black vine weevil in nursery ornamentals and soft fruit, other Coleoptera, western flower thrips in greenhouse ornamentals and chinch bugs in turf.

In a similarly preferred embodiment, said fungal microorganism is a strain of the species Isaria fumosorosea. Preferred strains of Isariafumosorosea are selected from the group consisting of Apopka 97, Fe9901, ARSEF 3581, ARSEF 3302, ARSEF 2679, IfB01 (China Center for Type Culture Collection CCTCC M2012400), ESALQ1296, ESALQ1364, ESALQ1409, CG1228, KCH J2, HIB-19, HIB-23, HIB-29, HIB-30, CHE-CNRCB 304, EH-511/3, CHE-CNRCB 303, CHE-CNRCB 305, CHE-CNRCB 307, EH-506/3, EH-503/3, EH-520/3, PFCAM, MBP, PSMB1, RCEF3304, PF01-N10 (CCTCC No. M207088), CCM 8367, SFP-198, K3, CLO 55, IfTS01, IfTS02, IfTS07, P1, If-02, If-2.3, If-03, Ifr AsC, PC-013 (DSMZ 26931), P43A, PCC, Pf04, Pf59, Pf109, FG340, Pfr1, Pfr8, Pfr9, Pfr10, Pfr11, Pfr12, Ifr531, IF-1106, 19602, 17284 ,103011 (Pat. US 4618578), CNRCB1, SCAU-IFCF01, PF01-N4, Pfr-612, Pf-Tim, Pf-Tiz, Pf-Hal, Pf-Tic.

It is most preferred that said Isaria fumosorosea strain is selected from Apopka 97 and Fe9901. A particularly preferred strain is APOPKA97.

Only few fungi with selective herbicidal activity are known, such as F2.1 Phoma macrostroma, in particular strain 94-44B (e.g. Phoma H and Phoma P by Scotts, US); F2.2 Sclerotinia minor, in particular strain IMI 344141 (e.g. Sarritor by Agrium Advanced Technologies); F2.3 Colletotrichum gloeosporioides, in particular strain ATCC 20358 (e.g. Collego (also known as LockDown) by Agricultural Research Initiatives); F2.4 Stagonospora atriplicis; or F2.5 Fusarium oxysporum, different strains of which are active against different plant species, e.g. the weed Striga hermonthica (Fusarium oxysproum formae specialis strigae).

In one embodiment, the fungus is selected from the group consisting of Isaria fumosorosea, Penicillium frequentans, Cladosporium cladosporioides, Cladosporium delicatum, Metarhizium spp., Beauveria bassiana, Beauveria brogniartii, Lecanicillium spp., Clonostachys rosea, Nomuraea rileyi, Trichoderma spp., Penicillium bilaii and Purpureocillium lilacinum.

In an especially preferred embodiment, the fungus is of the genus Trichoderma spp. or their respective teleomorphs, Hypocrea spp. Preferably said fungal strains belong to the species Trichoderma atroviride, Trichoderma asperellum, Trichoderma harzianum, Trichoderma viride, Trichoderma virens Trichoderma koningii, Trichoderma hamatum, Trichoderma gamsii, Trichoderma stromaticum, Trichoderma fertile, Trichoderma longibrachiatum or Trichoderma polysporum. As evident from the above lists of fungi active against different plant pests, species of Trichoderma spp. mainly act in plant health promotion and as fungicide against plant pathogens. Exemplary strains, belonging to said genus which are preferred are Trichoderma atroviride strain NMI no. V08/002387 (described in US8394623B2), strain NMI no. V08/002388, strain NMI no. V08/002389, strain NMI no. V08/002390, strain LC52 (e.g. Sentinel or Tenet from Agrimm Technologies Limited), strain CNCM 1-1237 (e.g. Esquive from Agrauxine, France), strain SC1 (e.g. Vintec from Bi-PA or Belchim, described in International Application No. PCT/IT2008/000196), strain B77 (e.g. T77 from Andermatt Biocontrol or Eco-77 from Plant Health Products), strain LUI32 (e.g. Tenet from Agrimm Technologies Limited), strain IMI 206040/ATCC 20476 (e.g. Binab TF WP from BINAB Bio-Innovation AB, Sweden), strain T11/IMI 352941/CECT 20498 (e.g. Tusal from Certis), strain SKT-1/FERM P-16510 (e.g. ECO-HOPE from Kumiai Chemical Industry Co), strain SKT-2/FERM P-16511, strain SKT-3/FERM P-17021, strain MUCL45632 (e.g. Tandem from Italpollina), strain WW10TC4/ATCC PTA 9707 (described in CA2751694A1), strain RR17Bc/ATCC PTA 9708, strain F11 Bab/ATCC PTA 9709; strain TF280 (described in CN107034146A), strain OB-1/KCCM 11173P (described in WO2012124863A1); Trichoderma harzianum strain KRL-AG2/ITEM 908/T-22/ATCC 20847 (e.g. Trianum-P from Koppert or PlantShield from BioWorks or Tricho D WP from Orius Biotecnologica), strain TH35 (e.g. Root-Pro from Mycontrol), strain T-39 (e.g. TRICHODEX and TRICHODERMA 2000 from Mycontrol), strain DB 103 (e.g. T-Gro 7456 from Dagutat Biolab, South Africa), strain DB 104 (e.g. Romulus from Dagutat Biolab, South Africa), strain TSTh20/ ATCC PTA-10317 (described in Application EP2478090A1), strain ESALQ 1306 (e.g. Trichodermil from Koppert), Rifai strain KRL-AG2 (e.g. BW240 WP from BioWorks), strain T78 (e.g. OffYouGrow Tric from Microgaia Biotech), strain from Trichopel (Agrimm Technologies), strain RR17Bc/ATCC PTA 9708 (described in CA2751694A1), strain ThLm1/NRRL 50846 (described in US20150033420A1), strain IBLF 006 (e.g. Ecotrich WP and Predatox SC from Ballagro Agro Tecnologia Ltda., Brazil), strain DSM 14944 (e.g. Agroguard WG and Foliguard from Live Systems Technology S.A, Colombia), strain 21 (e.g. Rootgard from Juanco SPS Ltd., Kenya), strain SF (e.g. Bio-Tricho from Agro-Organics, South Africa), strain IIHR-Th-2 (e.g. Ecosom-TH from Agri Life, India), strain MTCC5530 (described in US20120015806A1); Trichoderma virens (also known as Gliocladium virens) strain GL-21 (e.g. SoilGard by Certis, USA), strain G1-21, strain G1-3/ATCC 58678 (e.g. QuickRoots from Novozymes), strain DSM25764, strain G-41 (e.g. RootShieldPlus from BioWorks); Trichoderma viride strain TV1/MUCL 43093 (e.g. Virisan from Isagro), strain MTCC5532 (described in US20120015806A1), strain NRRL B-50520 (described in CN104203871A); Trichoderma polysporum strain IMI 206039/ATCC 20475/T-75 (e.g. Binab TF WP from BINAB Bio-Innovation AB, Sweden); Trichoderma stromaticum strain Ceplac 3550/ALF 64 (Tricovab from Ceplac, Brazil); Trichoderma asperellum strain kd (e.g. T-Gro from Andermatt Biocontrol or ECO-T from Plant Health Products), strain ICC 012/IMI 392716 (e.g. BIO-TAM and REMEDIER WP from Isagro Ricerca), strain B35 (Pietr et al., 1993, Zesz. Nauk. A R w Szczecinie 161: 125-137), strain BV10 (e.g. Tricho-Turbo from Biovalens), strain T34 (e.g. Asperello T34 Biocontrol from Biobest), strain T25/IMI 296237/CECT 20178 (e.g. Tusal from Certis), strain SKT-1 (e.g. Ecohope from Kumiai Chemical Industry Co.), strain URM 5911/SF04 (e.g. Quality WG from Laboratório de BioControle Farroupilha Ltda, Patos de Minas-MG, Brazil), strain H22 (e.g. TRICHOTECH WP from Dudutech); Trichoderma gamsii strain ICC 080 (e.g. BIO-TAM and REMEDIER WP from Isagro Ricerca), strain NRRL B- 50520 (described in WO2017192117A1); Trichoderma koningii strain SC164; Trichoderma hamatum strain TH382/ATCC 20765 (e.g. Floragard from Sellew Associates); Trichoderma fertile strain JM41R (e.g. TrichoPlus from BASF); Trichoderma longibrachiatum strain Mk1/KV966 (described in WO2015126256A1). Especially preferred fungal strains of the genus Trichoderma are Trichoderma atroviride strain CNCM I-1237, Trichoderma atroviride strain SC1 (e.g. Vintec from Bi-PA or Belchim, described in International Application No. PCT/IT2008/000196), and Trichoderma asperellum strain B35.

As mentioned above, the fungus is preferably produced as spores or conidia in the method of the present invention. More preferably, said cultivation is in the form of solid-state fermentation. Solid-state fermentation techniques are known in the art, see e.g. WO2005/012478 or WO1999/057239.

The present invention further relates to the use of a composite substrate as disclosed herein or a composite substrate produced according to the method disclosed herein for solid-state fermentation.

Further disclosed is the use of a composite substrate as disclosed herein or a composite substrate produced according to the method according to the invention for increasing spore yield during the cultivation of fungi.

The examples illustrate the present invention in a non-limiting fashion.

### EXAMPLE 1: PILOT-SCALE PRODUCTION OF FERMENTATION SUBSTRATE WITH POLYPROPYLENE AS THERMOPLASTIC

A fermentation substrate composed of polypropylen (PP) with a melting point of 165° C. (Total PP-H 9096) and native maize starch (Cornexo Maize Grits C1) was produced according to the following procedure.

The extrusion of a porous substrate suitable for fermentation composed of 70 wt% corn starch (>70 wt% amylopectin), 20 wt% wheat and rye bran for metabolic purposes and 10 wt% polypropylene homopolymer (PP-H, melting point 165° C., melt flow rate 25 at 2.16 kg, 10 min, 230° C.) was performed on a pilot scale co-rotating, parallel twin-screw extruder (model BC21) manufactured by Clextral with a length-to-diameter (L/D) ratio of 24, a shaft diameter D of 25 mm and nominal power of 8.3 kW. The material of plant origin was premixed using a ploughshare mixer and continuously fed to the intake zone of the extruder using a gravimetric screw feeder manufactured by Brabenderat a rate of 6 kg/h. The thermoplastic binder was fed to the extruder intake using a vibrating conveyor manufactured by Retsch at a rate of 0.7 kg/h. Dry materials were fed separately to the extruder intake due to large particle size ratios (~10) rendering mixing of both materials non-trivial. Along the direction of material flow the extruder shafts were configured with intake screw elements with high free volume (6D total length where D=shaft diamter), kneading elements for mixing (1D total length), screw elements with decreasing pitch length for plastification (6D total length), kneading element (1D total length), screw elements with 0.5D pitch length (10D). Water was injected into the shaft volume at a distance of 5D from the feeding section. The barrel had 5 temperature-controlled sections with temperatures set to 105° C., 140° C., 180° C. and 100° C. along the direction of material flow. A die plate for the extrusion of ring-shaped extrudates (inner diameter = 7 mm, outer diameter = 9 mm) was mounted to the outlet. As the extrudate emerged from the die they were cut using a rotary cutter equipped with two blades (model GR21) manufactured by Clextral. The process was operated continuously with a screw rotation speed of 350 rpm and a rotary cutter speed of 550 rpm. The maximum screw speed amounts to 680 rpm. A result a product temperature of 124° C., a product pressure of 125 bar and 28% torque relative to the maximum torque was measured. Accounting for a motor efficiency of 0.93 the above operation conditions represent a specific mechanical energy (SME) of 166 Wh/kg. Decreasing the screw speed, increasing the water inlet rate, decreasing the temperature, reducing the dry content mass rate or increasing the die hole diameter all were found to lower the expansion ratio of the extrudate and to decrease the porosity of the extrudate.

### EXAMPLE 2: LABORATORY-SCALE PRODUCTION OF FERMENTATION SUBSTRATE WITH ABS AS THERMOPLASTIC

A fermentation substrate composed of amorphous acrylonitrile-butadiene-styrene (Kumho Petrochemical Co., Kumho AB 750SW) with a heat deflection temperature of 80° C. according to ISO 75-2, native maize starch (Cornexo Maize Grits C1), talc nucleation agent and 99.5% commercial grade glycerol plasticizer can be produced according to the following procedure. The extrusion of the substrate suitable for fermentation is performed using the laboratory scale co-rotating, parallel twin-screw extruder (model Pharma 11 HME) manufactured by Thermo Scientific with a L/D ratio of 40, a shaft diameter of 11 mm and nominal power of 1.5 kW. The substrate formulation composed of 53 wt% native maize starch, 30 wt% ABS, 10 wt% glycerol, 6 wt% water, 1 wt% talc is prepared by pre-mixing glycerol and water, blending the liquid mixture with starch and finally mixing the wetted starch with ABS and talc. The mixture is moisture equilibrated for 3 h at 25° C. Extrusion of the starch/ABS blend takes place with a temperature profile of 90, 100, 120, 100° C. along the barrel axis as measured from feed to die. A 5 mm single hole die and a pelletizer are used to cut the substrate into a foamy cylindrical shape. Operating the extruder at a screw speed of 300 rpm results in a specific mechanical energy input of around 110 Wh/kg, a die pressure of 65 bar. The degree of substrate porosity is furthermore suitable for the following inoculation of the composite substrate with a microorganism to be cultivated.

1-25. (canceled)

26. Method for producing a microorganism comprising
(a) Providing a composite substrate which
i. was produced by melt extruding a thermoplastic which is mixed with a starch containing organic, granular or powdery, non-liquefiable growth medium which (A) comprises plant fibers or (B) is based on plants;
ii. comprises a melt extruded mixture of a thermoplastic and a starch containing organic, granular or powdery, non-liquefiable growth medium which (A) comprises plant fibers or (B) is based on plants; or
iii. was obtained from a method comprising
I. Mixing of at least one thermoplastic with a starch containing, organic, granular or powdery, non-liquifiable growth medium;
II. Melt extruding the mixture obtained from I. into a desired shape;
(b) Inoculating the composite substrate with a microorganism to be cultivated; and
(c) Incubating the composite substrate obtained from step (b) under controlled conditions.

27. Method according to claim 26, characterized in that said extrusion takes place in a temperature range of between 120 and 220° C.

28. Method according to claim 26, characterized in that said extrusion takes place at a pressure of between 2.5 and 35 MPa.

29. Method according to claim 26, characterized in that the thermoplastic is selected from the group consisting of polyolefins, polyvinylchloride, polyester, polyamide, polystyrene, polyurethane, a derivative of any of the foregoing and copolymers of any of the foregoing.

30. Method according to claim 26, characterized in that the shape is selected from the group consisting of a polyhedron, a sphere or a part thereof, a torus, a cylinder, a cone, an ellipsoid, a paraboloid and a hyperboloid.

31. Method according to claim 26, characterized in that the shape in its longest dimension has a diameter of between 2 and 50 mm.

32. Method according to claim 26, characterized in that the ratio of thermoplastic:organic growth medium is between 5:95 and 80:20.

33. Method according to claim 26, characterized in that the organic growth medium is selected from micro- and macronutrients and mixtures thereof.

34. Method according to claim 26, characterized in that the organic growth medium is selected from the group consisting of:
(A) cereals, mixtures of cereals, parts of cereal grains, ground timber, other plant parts and food waste comprising polysaccharides and mixtures of any of the foregoing; or
(B) wheat, rye, oat, rice, barley, maize, triticale, sorghum, soybean, and mixtures thereof.

35. Method according to claim 34, wherein a cereal of (A) or (B) is present as malt.

36. Method according to claim 34, characterized in that the cereal or other plant part is coarsely ground or pulverized.

37. Method according to claim 26, characterized in that said thermoplastic is present as granulate or powder.

38. Method according to claim 26, wherein the composite substrate:
(A) is porous;
(B) is a substrate for solid-state fermentation of microorganisms; or
(C) comprises 5-20 parts thermoplastic 30-95 parts of a starch containing, organic, granular or powdery, non-liquifiable growth medium.

39. Method according to claim 26, wherein the microorganism is a fungus, a yeast or a bacterium.

40. Method according to claim 39, wherein the fungus is selected from the group consisting of Isaria fumosorosea, Penicillium frequentans, Cladosporium cladosporioides, Cladosporium delicatum, Metarhizium spp., Beauveria bassiana, Beauveria rogniartii, Lecanicillium spp., Clonostachys rosea, Nomuraea rileyi, Trichoderma spp., Penicillium bilaii and Purpureocillium lilacinum.

41. Method according to claim 39, wherein the fungus is produced as spores or conidia.

42. Method according to claim 39, characterized in that the said cultivation is in the form of solid-state fermentation.

43. Composite substrate, which:
i. is produced by melt extruding a thermoplastic which is mixed with a starch containing organic, granular or powdery, non-liquefiable growth medium which (A) comprises plant fibers or (B) is based on plants; or
ii. comprises a melt extruded mixture of a thermoplastic and a starch containing organic, granular or powdery, non-liquefiable growth medium which (A) comprises plant fibers or (B) is based on plants.

44. Composite substrate according to claim 43, wherein the composite substrate:
(A) is porous;
(B) is a substrate for solid-state fermentation of microorganisms; or
(C) comprises 5-20 parts thermoplastic 30-95 parts of a starch containing, organic, granular or powdery, non-liquifiable growth medium.

45. A method of using a composite substrate as defined in claim 26 for:
(a) solid-state fermentation; or
(b) increasing the yield during the cultivation of fungi.
